(19) United States
(12) Patent Application Publication
(10) Pub. No.: US 2003/0162727 A1
(43) Pub. Date: Aug. 28, 2003

(54) Use of cyp1b1inhibitors for treating cancer

(76) Inventors: Graeme Ian Murray, Aberoeen (GB); William Thomas Melvin, Aberdeen (GB); Morag McFadyen, Aberdeen (GB)

Correspondence Address: QUINE INTELLECTUAL PROPERTY LAW GROUP, P.C., P O BOX 458, ALAMEDA, CA, 94501, US

(21) Appl. No.: 10/203,484

(22) PCT Filed: Feb. 9, 2001
(86) PCT No.: PCT/GB01/00548

(30) Foreign Application Priority Data
Feb. 9, 2000 (GB) 0002835.7

(51) Int. Cl. A61K 31/7048; A61K 31/704; A61K 31/353; A61K 31/337; A61K 31/137; A61K 31/525; A61K 31/015

(52) U.S. Cl. 514/27; 514/449; 514/456; 514/34; 514/651; 514/251; 514/765

(57) ABSTRACT

CYP1B1 proteins and their role in metabolising or inactivating anti-cancer drugs is disclosed, together with compositions for treating cancer comprising a substance capable of inhibiting CYP1B1 protein and an anti-cancer drug (e.g. docetaxel, paclitaxel, flutamide, tamoxifen, mitoxantrone, doxorubicin or daunomycin).

### FIELD OF THE INVENTION

The present invention relates to CYP1B1 proteins and their role in cancer, and more particularly to the use of inhibitors of CYP1B1 proteins to ameliorate the inactivation of anti-cancer drugs by CYP1B1 present in cancer cells.

### BACKGROUND OF THE INVENTION

The cytochromes P450 are a multi-gene family of constitutive and inducible enzymes which have a central role in the oxidative metabolic activation and detoxification of a wide range of xenobiotics and several groups of endogenous compounds active in cell regulation and cell signalling, including arachidonic acid, steroid hormones and fatty acids. The major families of P450 involved in xenobiotic metabolism each consist of several individual forms with different regulatory mechanisms and substrate specificities. The majority of P450s are primarily expressed in liver, although individual P450 forms are also expressed in specific extra-hepatic tissues including small intestine, kidney and lung.

The human CYP1 gene family, which is one of the major P450 families involved in the metabolism of xenobiotics, is now known to consist of three individual forms classified into two sub-families. The CYP1A subfamily contains two highly homologous and well characterised but distinct members, CYP1A1 and CYP1A2. CYP1A1 is an inducible P450 expressed primarily in extrahepatic tissues while CYP1A2 is a major form of P450 that is constitutively expressed in liver. There is also a second human CYP1 subfamily which contains one member, CYP1B1. This P450 is dioxin-inducible and sequence analysis of CYP1B1 shows 40% homology with both CYP1A1 and CYP1A2. Although CYP1B1 is assigned to the CYP1 family on the basis of its sequence, it is structurally and functionally distinct from both CYP1A1 and CYP1A2.

WO97/12246 (University of Aberdeen) discloses that CYP1B1 is present in a range of tumour cells and proposes the use of this enzyme as a marker for the diagnosis of cancer. This application further discloses therapies for the treatment of cancer based on the presence of CYP1B1 in tumour cells. In one embodiment, the application proposes a selective therapy employing drugs which are activated by CYP1B1 present in tumour cells, converting a prodrug into a cytotoxic form capable of killing tumour cells. In other embodiments, WO97/12246 proposes the use of CYP1B1 present in tumour cells as a marker to guide a therapeutic compound selectively to the cells, e.g. by conjugating a drug to a moiety which is capable of specifically recognising the CYP1B1. WO97/12246 further suggests that CYP1B1 may be involved in providing an essential function for tumour cells in inactivating endogenous anti-tumour compounds such as 2-methoxyestradiol. In view of this, the application proposes reducing endogenous CYP1B1 levels in tumour cells, e.g. by using antisense RNA or suicide inhibitors to inhibit CYP1B1 production.

WO00/56773 (University of Aberdeen) relates to the fragments of CYP1B1 for use in raising antibodies capable of specifically binding CYP1B1, and the use of such antibodies for the diagnosis or treatment of cancers linked to enhanced CYP1B1 expression.

### SUMMARY OF THE INVENTION

Broadly, the present invention is based on the finding that the presence of CYP1B1 in tumour cells contributes to the resistance of tumour cells to anti-cancer drugs. This opens up the possibility of enhancing the effectiveness of cancer therapies employing these anti-cancer drugs by inhibiting CYP1B1 or reducing its level in tumour cells thereby ameliorating the effect of the CYP1B1 in inactivating or metabolising the drugs.

CYP1B1 is a member of the cytochrome P450 superfamily of enzymes. There have been some reports in the prior art that a number of the other P450 family members are involved in a biotransformation of anti-cancer drugs used to treat a variety of cancers. Cytochrome P450 mediated metabolism generally leads to inactivation or reduced activity of the drug, whereas cyclophosphamide, an inactive pro-drug, must firstly undergo a 4-hydroxylation reaction by cytochrome P450 enzymes before becoming cytotoxic. However, as set out above, there is not a close relationship between CYP1B1 and other P450 family members in terms of their structure or the substrates on which the enzymes act. Accordingly, the finding that CYP1B1 causes drug resistance is not predictable based on the results obtained with other P450 enzymes.

Ovarian cancer causes the greatest number of deaths from gynaecological malignant disease in the developed world. The lack of symptoms in the early stages of ovarian cancer means that up to 80% of newly diagnosed patients will have disease that is advanced, where complete surgical resection is not possible and with an overall five year survival of only 30%. Early stage ovarian carcinoma has a good prognosis with 80% five year survival and generally chemotherapy is not recommended, whereas adjuvant chemotherapy with a platinum-based regimen is advocated for patients with advanced disease. The results disclosed herein confirm that CYP1B1 expression in ovarian cancer shows a strong correlation with patient survival for treatment with paclitaxel. The recent introduction of the taxane, paclitaxel, for the treatment of ovarian cancer may result in a better progression-free and overall survival. Docetaxel, a semi-synthetic taxane, is currently being evaluated for use in the treatment of ovarian cancer. Some of the results disclosed herein examine the ability of CYP1B1 to alter the cytotoxic profile of fourteen anti-cancer drugs commonly used as first line treatment in solid tumours.

In the studies disclosed herein, a Chinese hamster ovary cell line expressing human cytochrome P450 CYP1B1 was used to evaluate the cytotoxic profile of several anti-cancer drugs (docetaxel, paclitaxel, cyclophosphamide, doxorubicin, 5-fluorouracil (5FU), cisplatin and carboplatin) commonly used clinically in the treatment of cancer. The MTT (3-[4,5-dimethylthiazol-2yl]-2,5-diphenyltetrazolium bromide) assay, was used to determine the levels of cytotoxicity. The key finding of this study was that on exposure to docetaxel and paclitaxel, a decrease in sensitivity towards the cytotoxic effects of the drug was observed in the cell line expressing CYP1B1 compared to the parental cell line. This effect was particularly marked for docetaxel (p=0.03), while the lesser result obtained with paclitaxel may be due to the particular cell line used in the assay or because the metabolic product of paclitaxel retained some cytotoxicity to the cells. Further, this difference in cytotoxicity was reversed by co-incubation of the cells with both docetaxel and the cytochrome P450 CYP1 inhibitor α-naphthoflavone (ANF). This study is the first to indicate, that the presence of CYP1B1 in cells decreases their sensitivity to the cytotoxic effects of a specific anti-cancer drug. Further studies with kidney tumours showed that addition of the CYP1B1 inhibitor α-naphthoflavone inhibited metabolism of EROD by CYP1B1 in the tumour cells.

Several cytochrome P450 enzymes are involved in the metabolism of a range of anti-cancer drugs, such as cyclophosphamide, paclitaxel and docetaxel [9-14]. Cytochrome P450 mediated metabolism usually results in reduced activity or inactivation of the anti-cancer drugs but in some cases bioactivation to a more cytotoxic metabolite occurs. One example of detoxification of anti-cancer drugs can be shown by the taxanes. The major pathway of metabolism of paclitaxel, an anti-cancer drug used in the treatment of breast, ovarian and non-small cell lung cancer, is catalysis by CYP2C8 and involves the hydroxylation of position 6 on the taxane ring [15]. The metabolite 6-hydroxytaxol is 30 fold less cytotoxic than the parent compound paclitaxel [16], and 6-hydroxytaxol is further metabolised by CYP3A4 [13]. Docetaxel, a semi-synthetic taxane, currently undergoing phase II and phase III trials for use in first-line therapy of ovarian cancer is metabolised by CYP3A to apparently less cytotoxic metabolites [12,13].

Accordingly, in a first aspect, the present invention provides the use of a substance capable of inhibiting CYP1B1 protein and an anti-cancer drug for the preparation of a medicament for the treatment of cancer.

Preferably, the substance capable of inhibiting CYP1B1 protein inhibits the activity of CYP1B1 enzyme in metabolising the anti-cancer drug or a pro-drug form thereof, or a metabolic product of the anti-cancer drug or pro-drug, thereby inactivating, detoxifying or otherwise modifying it so that it loses some or all of its anti-cancer activity (e.g. cytotoxicity to cancer cells), or in the case of a pro-drug, the capacity to be converted to active drug. Examples of CYP1B1 inhibitors are well known in the art and include flavones and flavonoids such as α-naphthoflavone (ANF), accacatin, diosmetin, hesperetin and homoeriodictyol, and 2-ethynylpyrene. See, for example, Doostdar et al, Toxicology, 144:31-38, 2000 and Shimada et al, Chem. Res. Toxicol., 11:1048-1056, 1998. Preferred inhibitors are selective, that is they inhibit CYP1B1 while having a reduced inhibitory effect, or more preferably substantially no effect, on the function of endogenous enzymes present in normal cells. In addition to using known inhibitors, the skilled person can readily screen libraries of compounds to look for further inhibitors for use in accordance with the present invention.

The anti-cancer compound may be a drug or a pro-drug and is preferably a substrate of CYP1B1. Examples of suitable anti-cancer drugs for use in conjunction with the CYP1B1 inhibitors include docetaxel, paclitaxel, flutamide, tamoxifen, mitoxantrone, doxorubicin or daunomycin, and more especially taxanes such as docetaxel and paclitaxel. The results disclosed herein show that all of these drugs inhibit the action of CYP1B1 on its substrate EROD, and further experiments with paclitaxel and docetaxel confirm this result in cell assays. The CYP1B1 inhibitor and the anti-cancer agents may be formulated together in a composition or separately for simultaneous or sequential administration.

Preferably, the types of cancer treatable using the present invention are those characterised by the presence of CYP1B1 in tumour cells and more especially, the presence of CYP1B1 at an elevated level. Examples of such types of cancer include breast cancer, kidney cancer, colorectal cancer, prostate cancer, liver cancer or ovarian cancer.

Accordingly, in a further aspect, the present invention provides a composition comprising a substance capable of inhibiting CYP1B1 protein and an anti-cancer drug, in combination with a physiologically acceptable carrier.

In a further aspect, the present invention provides a kit comprising:

(a) in a first container, a substance capable of inhibiting CYP1B1 protein; and

(b) in a second container, an anti-cancer drug; wherein the substances are formulated with a physiologically acceptable carrier and are for simultaneous or sequential administration. The kit may also include instructions for administering the components of the kit.

In a further aspect, the present invention provides a method for treating cancer, the method comprising administering to a patient in need a combination of a substance capable of inhibiting CYP1B1 protein and an anti-cancer drug.

In a further aspect, the present invention provides a method of screening for CYP1B1 inhibitors for use in combination with anti-cancer drugs, the method comprising:

(a) contacting a candidate substance with CYP1B1 protein under conditions where the candidate substance and CYP1B1 can interact;

(b) measuring the activity of the CYP1B1 protein and comparing the value obtained to standards; and

(c) selecting candidate compounds which have the effect of inhibiting CYP1B1.

In one embodiment, the method involves contacting CYP1B1 with a candidate compound and a substrate of CYP1B1 (e.g. EROD) and the effect of the candidate compound can be determined by monitoring the effect of the candidate compound in inhibiting the breakdown of the substrate by CYP1B1.

Optionally, the method may comprise the additional step of testing the candidate compounds, e.g. in combination with CYP1B1 and one or more anti-cancer drugs, to determine the effect the candidate inhibitor has in reducing the effect of CYP1B1 in inactivating or metabolising the anti-cancer drug. Examples of assays include the in vitro interaction assays described below and cell based assays measuring the effect of the inhibitors in reducing the loss of cytotoxicity of the anti-cancer drugs caused by CYP1B1 action.

In carrying out these methods, it may be convenient to screen a plurality of candidate compounds, e.g. as present in a library, at the same time, e.g. by contacting a mixture of different candidate compounds with the interacting peptides, and then in the event of a positive result resolving which member of the mixture is active. These techniques are used in high throughput screening (HTS) to increase the numbers of compounds, e.g. resulting from a combinatorial chemistry program or present in a library derived from a natural source material.

The precise format of the assays of the invention may be varied by those of skill in the art using routine skill and knowledge. For example, interaction between substances may be studied in vitro by labelling one with a detectable label and bringing it into contact with the other which has been immobilised on a solid support. The amount of candidate substance or compound which may be added to an assay of the invention will normally be determined by trial and error depending upon the type of compound used. Typically, from about 0.01 to 100 nM concentrations of putative inhibitor compound may be used, for example from 0.1 to 10 nM.

In a further aspect, the present invention provides a method of determining the diagnosis, prognosis or responsiveness to treatment of a patient having ovarian cancer, the method comprising determining a presence or amount of CYP1B1 protein in a sample from a patient comprising ovarian cancer cells and correlating the presence or amount to control values. Examples of assays for use in this aspect of the invention are disclosed on WO97/12246 and antibodies suitable for use in immunoassays are disclosed in WO00/56773.

Embodiments of the present invention will now be described by way of example and not limitation with reference to the accompanying figures.

### BRIEF DESCRIPTION OF THE FIGURES

FIG. 1. Survival curves of cells treated with A, docetaxel; B, paclitaxel; C, cyclophosphamide; D, doxorubicin; E, 5-FU; F, carboplatin and G, cisplatin. Parental V79MZ cells and those transfected with CYP1B1 (V79MZh1B1) were incubated with increasing concentrations of the appropriate drug for 24 h. Cell viability was then determined by the MTT assay and the percentage of surviving cells relative to the respective controls (cells treated with solvent only), was calculated for each drug concentration. There was significantly different cytotoxicity for docetaxel (A) in CYP1B1 expressing and non-expressing cell lines and a lesser effect was observed with paclitaxel, perhaps because the action of CYP1B1 resulted in a product which retained some cytotoxicity. There was no cytotoxicity observed in cells exposed to cyclophosphamide (C). Each 96 well plate allowed eight concentrations of the appropriate drug per plate, with eight replicates (i.e. eight separate wells) per concentration. (i.e. 8 measurements for each concentration of drug per plate, There were three triplicate plates per experiment resulting in a total of 24 measurements of absorbance per concentration of drug). The standard deviation was less than 5% of the mean absorbance for all drugs used, and at each concentration of drug.

FIG. 2 Survival curve of cells treated with docetaxel in the presence or absence of the cytochrome P450 inhibitor ANF. Parental V79MZ cells and those transfected with CYP1B1 (V79MZh1B1) were incubated with increasing concentrations of docetaxel for 24 hr. ANF was added at the following concentration 1, 10, or 100 μM to the transfected V79MZh1B1 cells. Cell viability was then determined by the MTT assay and the percentages of surviving cells, relative to the respective solvent controls, were calculated. ANF at a concentration of 100 μM totally abolished the differential cytotoxicity observed in the V79MZh1B1 transfected cells. ANF, itself, exhibited no cytotoxic effects on the parental (V79MZ) and CYP1B1 (V79MZh1B1) expressing cell line, at all of the concentrations used.

FIG. 3. Survival of patients treated with docetaxel as part of their anti-cancer drug regimen and classified according to CYP1B1 status of their primary tumours. The presence of strong/moderate CYP1B1 immunoreactivity results in a poorer overall survival compared with weak or absent CYP1B1 immunoreactivity.

### DETAILED DESCRIPTION

Pharmaceutical Compositions

The compounds described herein or their derivatives can be formulated in pharmaceutical compositions, and administered to patients in a variety of forms, in particular to treat cancer, and more especially breast cancer, colorectal cancer, prostate cancer, liver cancer or ovarian cancer.

Pharmaceutical compositions for oral administration may be in tablet, capsule, powder or liquid form. A tablet may include a solid carrier such as gelatin or an adjuvant or an inert diluent. Liquid pharmaceutical compositions generally include a liquid carrier such as water, petroleum, animal or vegetable oils, mineral oil or synthetic oil. Physiological saline solution, or glycols such as ethylene glycol, propylene glycol or polyethylene glycol may be included. Such compositions and preparations generally contain at least 0.1 wt % of the compound.

Parental administration includes administration by the following routes: intravenous, cutaneous or subcutaneous, nasal, intramuscular, intraocular, transepithelial, intraperitoneal and topical (including dermal, ocular, rectal, nasal, inhalation and aerosol), and rectal systemic routes. For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, solutions of the compounds or a derivative thereof, e.g. in physiological saline, a dispersion prepared with glycerol, liquid polyethylene glycol or oils.

In addition to one or more of the compounds, optionally in combination with other active ingredient, the compositions can comprise one or more of a pharmaceutically acceptable excipient, carrier, buffer, stabiliser, isotonicizing agent, preservative or anti-oxidant or other materials well known to those skilled in the art. Such materials should be non-toxic and should not interfere with the efficacy of the active ingredient. The precise nature of the carrier or other material may depend on the route of administration, e.g. orally or parentally.

Liquid pharmaceutical compositions are typically formulated to have a pH between about 3.0 and 9.0, more preferably between about 4.5 and 8.5 and still more preferably between about 5.0 and 8.0. The pH of a composition can be maintained by the use of a buffer such as acetate, citrate, phosphate, succinate, Tris or histidine, typically employed in the range from about 1 mM to 50 mM. The pH of compositions can otherwise be adjusted by using physiologically acceptable acids or bases.

Preservatives are generally included in pharmaceutical compositions to retard microbial growth, extending the shelf life of the compositions and allowing multiple use packaging. Examples of preservatives include phenol, meta-cresol, benzyl alcohol, para-hydroxybenzoic acid and its esters, methyl paraben, propyl paraben, benzalconium chloride and benzethonium chloride. Preservatives are typically employed in the range of about 0.1 to 1.0% (w/v).

Preferably, the pharmaceutical compositions are given to an individual in a “prophylactically effective amount” or a “therapeutically effective amount” (as the case may be, although prophylaxis may be considered therapy), this being sufficient to show benefit to the individual. Typically, this will be to cause a therapeutically useful activity providing benefit to the individual. The actual amount of the compounds administered, and rate and time-course of administration, will depend on the nature and severity of the condition being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980. By way of example, the compositions may be administered to patients in dosages of between about 0.01 and 100 mg of active compound per kg of body weight, and more preferably between about 0.5 and 10 mg/kg of body weight.

Materials and Methods

Tissue

Samples of ovarian cancer (n=172) submitted to the Department of Pathology, University of Aberdeen for diagnosis, over a five year period (1993-1998), were used in this study, with ethical approval from the Joint Grampian Health Board and University of Aberdeen Research Ethics Committee. Of the 172 cases of ovarian cancer, 167 cases were of the primary ovarian tumour, 43 of these cases had samples of both primary and metastatic disease, while 5 cases had samples of metastatic deposits with no corresponding ovarian tumour for investigation, (i.e. a total of 48 cases of metastatic disease and 167 cases of primary ovarian tumour). In 49 cases contralateral normal ovary was also submitted for histopathological examination and available for study. All the tissue samples had been fixed in 10% neutral buffered formalin for 24 hours and then routinely processed to paraffin wax. The diagnosis of ovarian cancer was performed with hematoxylin and eosin stained sections using standard histopathological criteria by a consultant histopathologist with special interest in gynaecological pathology. The tumours were graded according to criteria described by FIGO. The median age of patients in this study was 63 years with an age range from 30-89 years. Information on therapeutic treatment and clinical outcome was available for 170 patients. The following anti-cancer drugs (cisplatin, carboplatic, cyclophosphamide, paclitaxel, and docetaxel) were used to treat the patients. Most patients received either cisplatin or carboplatin. The other three drugs were usually given in combination with a platinum based drug. Following diagnosis, the disease status of patients was assessed at regular intervals by two gynaecological oncologists with the median overall survival of patients being 17 months.

Localisation of CYP1B1 in Ovarian Cancer

Immunohistochemical detection of CYP1B1 with a monoclonal antibody to CYP1B1 was performed using a tyramine signal amplification method as described previously [3]. Sites of immunoreactivity were demonstrated colorimetrically with diaminobenzidine and Hydrogen peroxide (Liquid DAB plus, Dako Ltd High Wycombe, Bucks, UK). Positive control tissue was sections of breast cancer, which had been previously shown to contain CYP1B1 by immunohistochemistry [3], the negative control used Tris buffered saline (TBS) in place of the primary monoclonal antibody. To establish the presence or absence of CYP1B1 and its distribution, intensity and cellular localisation, the sections were examined using bright field light microscopy by two independent observers. CYP1B1 immunoreactivity in the tumours was assessed as strong (3), moderate (2), weak (1), or negative (0). Tumours exhibiting CYP1B1 immunoreactivity in more than 5% of the cell were considered as positive.

Cell Lines and Cell Culture

A Chinese hamster ovary fibroblast cell line (V79MZ) and a clone expressing human CYP1B1 (V79MZh1B1) [21], were grown at 37° C., 5%CO₂ and at 90% saturated humidity, in DMEM (Dulbecco's modified eagles medium) high glucose type supplemented with 1 mM sodium pyruvate, 10% fetal calf serum, 100 U penicillin/ml and 100 μg streptomycin/ml. Neither cell line expresses endogenous P450s, although cytochrome P450 reductase is present in both cell lines [22,23,24]. The parental and CYP1B1 expressing cell lines double in cell number every 10-12 h [23] and were sub-cultured at 1:50 ratio (i.e. 1 ml of cells to 50 ml of fresh media) every 4-5 days. The cells were not allowed to reach confluency at any time, to ensure optimal cell physiological conditions and maximal cytochrome P450 activity [23]. Cells were routinely used at 3^{rd}-5^{th} passage for all experiments [21].

Immunoblotting of CYP1B1 Protein in V79 Total Cellular Homogenate

Both cell lines (V79MZ and V79MZh1B1) were grown to 60-80% confluence and a total cellular homogenate from each prepared. Cellular protein was determined according to the method of Bradford [25]. Samples of cellular homogenates were then resolved by SDS-PAGE on a 10% polyacrylamide gel using a Hoefer SE 600 (Amersham Pharmacia Biotech) vertical gel electrophoresis system. This was followed by transfer to nitrocellulose membrane (Hybond-ECL, Amersham Pharmacia Biotech). Sites of immunoreactivity were detected with a monoclonal antibody to human CYP1B1 [3]. This antibody was raised against a 15 amino acid peptide corresponding to amino acid residues 437-451 of the human CYP1B1 protein [3]. CYP1B1 immunoreactivity was visualised by an enhanced chemiluminescence detection system (Amersham Pharmacia Biotech). Microsomes prepared from human lymphoblastoid cells which have been transfected to stably express human CYP1B1 (Gentest) were used as the positive control [26].

Cell Viability Assay

Paclitaxel, cyclophosphamide, doxorubicin 5-FU, cisplatin, carboplatin and ANF were purchased from Sigma. Docetaxel was obtained from Rhone-Poulenc Rorer. Optimal growth conditions were established. The same conditions were used for both cell lines; V79MZ and V79MZh1B1 cells at 60-80% confluence were harvested and seeded at 0.5-1×10³ cells per well in 96 well culture plates. Cells were grown for 48 hours, media was then removed and each drug added at increasing concentrations in the appropriate solvent (0.1% ethanol for docetaxel, paclitaxel, cyclophosphamide and carboplatin, 0.1% DMSO for cisplatin, sterile water for 5-FU and doxorubicin). Cells treated with solvent alone acted as a negative control.

Enzyme inhibition studies were undertaken by co-administration of docetaxel and the known P450 CYP1 inhibitor ANF (Sigma) [27] at different concentration in the media. Stock solutions of ANF (1 mM, 10 mM and 100 mM) were dissolved in DMSO and added to the media to give final concentrations of 1 μM, 10 μM and 100 μM ANF (in each case the final concentration of DMSO in media was 0.1%). Following 24 h exposure to each drug (with or without inhibitor), the media were removed and replaced with fresh media without drug. In this study, cells were then grown for three doubling times (36 h) with the media changed at 24 hr. Cell viability was then assessed using the MTT assay, which is comparable to using a clonogenic assay [23,28,29]. Media were removed from the wells and replaced with 200 μl of fresh media, followed by addition of 50 μl of MTT solution (50 mg/ml of MTT (Sigma) in sterile PBS), the cells were incubated at 37° C. in a humidified atmosphere with 5%CO₂/95% air for 4 h. The MTT containing media was then removed and 200 μl of DMSO plus 25 μl of glycine buffer (0.1 M glycine/0.1 M NaCl pH 10.5) added to the cells in each well. This procedure overcomes any effect that cell density or culture medium may have on the absorption spectrum [28]. The absorbance of the formazan produced by the viable cells was measured once for each well at 540 nm on a Labsystems EMS microplate reader (Life Sciences International). To calculate the cell viability cells treated with solvent alone were assigned a value of 100% absorbance indicating zero cytotoxicity, i.e. 100% viability. The cytotoxic profile of each drug was evaluated in triplicate 96 well plates. Each plate included several controls (media only, cells only and cells treated with solvent alone). The 96 well plate format allowed eight concentrations of the appropriate drug per plate, with eight replicates (i.e. eight separate wells) per drug concentration, i.e. 8 measurements of absorbance for each concentration of drug per plate. There were three triplicate plates per experiment resulting in a total of 24 measurements of absorbance per concentration of drug. Plate reader variability was found to be negligible.

P4501B1 has a Role in Metabolism of Anticancer Agents

Recent immunohistochemical analysis of P4501B1 protein in a variety of solid tumours identified tumour-specific enzyme expression (Murray et al, 1998). This provides a mechanism of resistance to currently used anticancer agents, as well as, an approach for targeted prodrug therapy. 7-ethoxyresorufin deethylase activity (EROD) was used to define interactions with cytochrome 1B1 (CYP1B1). 7-Ethoxyresorufin is a known substrate for P4501B1. EROD activity was used to screen the binding affinity of anticancer agents and test compounds with CYP1B1. For this purpose, inhibition constants (Ki) have been calculated using in vitro kinetics from CYP1B1-mediated EROD activity. EROD activity and assay has been described previously by various authors (Lubet et al., 1985, Arch. Biochem. Biophys. 238: pp 43-48; Gentest Corporation, 6 Henshaw St., Woburn, Mass. 01801 USA, data sheet given with CYP1B1, catalogue number P220). 7-Ethoxyresorufin is enzymatically deethylated to a product called resorufin. The appearance of resorufin was monitored by a fluorescence detector set at 550 nm and 582 nm (excitation and emission wavelengths). Various concentrations of 7-ethoxyresorufin (0.05, 0.1 and 0.5 μM) as well as various concentrations of anti-cancer agents and test (control) compounds (depending on the efficiency of the inhibition) were added to the incubation system. Incubations contained: phosphate buffer at pH 7.4, 7-ethoxyresorufin, CYP1B1 Supersomes from Gentest Co (catalogue number P220) and nicotine adenine dinucleotide phosphate (NADP) as cofactor.

Biotransformation Studies

Flutamide and paclitaxel (taxol) were tested as potential substrates of the CYP1B1. The results show that both drugs are biotransformed by CYP1B1, as metabolites were produced by CYP1B1 incubation but not control incubations.

Kidney Tumour Assays

The effect of a CYP1B1 inhibitor was confirmed in an assay using kidney tumour samples. A buffer and NADP regenerating system was allowed to equilibrate at 37° C. and EROD (5 μM final concentration) was then added. This was followed by the addition of either P450 supersomes (control) or the appropriate tumour sample (1 mg). Supersomes were incubated for 15 minutes and tumour samples for 40 minutes. The CYP1B1 inhibitor α-naphthoflavone was added to some samples just prior to the addition of EROD and P450 sample.

Statistics

Statistical analysis was performed using both Statistics for Windows 95 and SPSS version 7.5 for Windows 95.

Results

Localisation of CYP1B1 in Ovarian Cancer

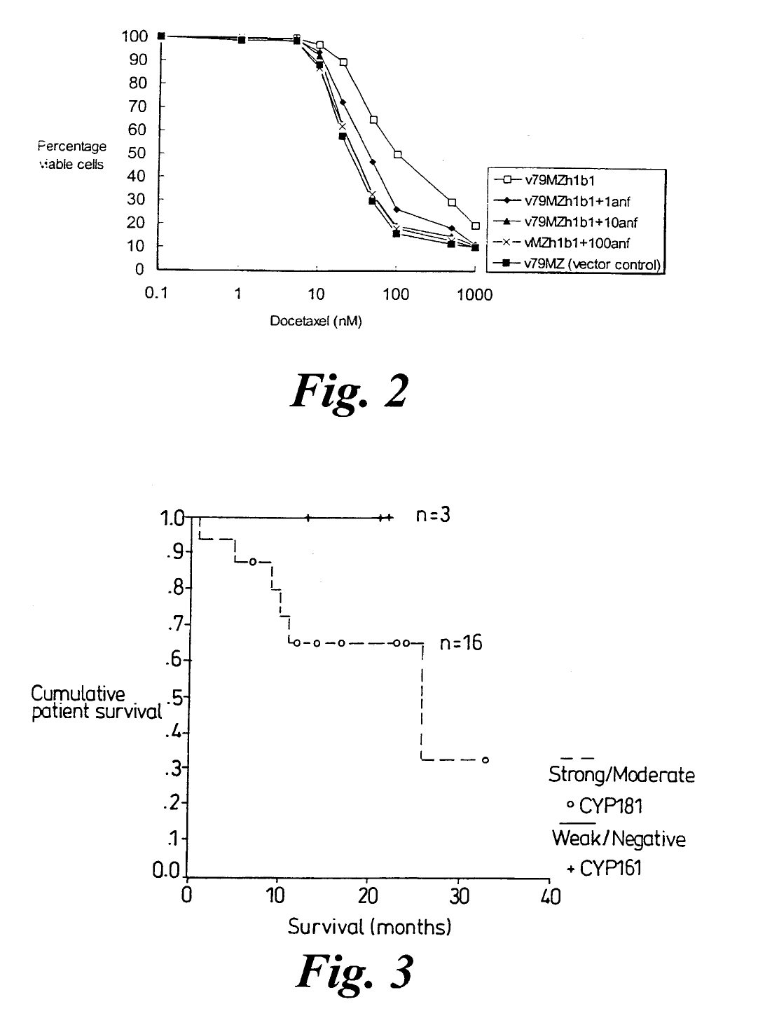

CYP1B1 immunoreactivity was identified in the majority (153/167; 92%) of primary ovarian cancer sections and was specifically localised to the cytoplasm of tumour cells. There was no detectable CYP1B1 expression in any of the normal ovarian tissue samples. In a high percentage of the ovarian cancers there was either strong (85/167; 50.9%) or moderate (39/167; 23.4%) immunoreactivity for CYP1B1 (Table 4). In addition, the presence of CYP1B1 was observed in the majority (45/48; 95%) of metastatic deposits with a high proportion showing moderate (22/48;45.8%) to strong (18/48;37.5%) immunoreactivity (Table 5). A similar level of CYP1B1 expression was exhibited for the different histological subtypes in both primary and metastatic tumour (Tables 4 and 5). In the cases where both primary ovarian tumour and metastatic deposits were available, a significant correlation for CYP1B1 expression (p=0.05 Spearman correlation test) was observed). The presence of moderate to strong CYP1B1 in the tumours of the subset of 19 patients who had received docetaxel as part of their anti-cancer drug regime had an adverse effect on overall survival (FIG. 3). The presence of CYP1B1 had no influence on the survival of patients who had received other anti-cancer drug regimes.

In parallel with the in vitro studies of CYP1B1 on the cytotoxic profile of the anti-cancer drugs, a comprehensive investigation was conducted into the presence of CYP1B1 in primary ovarian tumour and metastatic deposits and the influence it has on the overall survival of patients on different therapeutic regimens. A monoclonal antibody to CYP1B1 was used to demonstrate the localisation of CYP1B1 to ovarian tumour cells and lack of expression in normal ovarian tissue. The over-expression of CYP1B1 was observed in all histological subtypes of epithelial ovarian cancer and at high frequency (>85%). This finding is in agreement with a previous study, which utilised a polyclonal antibody to CYP1B1 to demonstrate the over-expression of CYP1B1 in a small number (7/7) of ovarian serous adenocarcinomas (7). A high frequency of CYP1B1 over-expression was also observed in the majority of metastatic deposits in this current study, and was highly correlated with paired primary tumour. These findings also support the concept of CYP1B1 being a molecular target for the development of new treatment approaches to ovarian cancer.

During the time period in which patients on this study were diagnosed (1993-1998), considerable changes were observed in the chemotherapeutic regimen used for the treatment of ovarian cancer. Prior to 1994 the main treatment of choice was a cisplatin/cyclophosphamide based-regimen (19). Although a platinum agent is still regarded as essential for primary treatment, the less toxic agent carboplatin is now the platinum agent of choice. Following the introduction of paclitaxel to oncological practice, the principal first line therapy for ovarian cancer is currently carboplatin plus paclitaxel (19). Docetaxel, is currently under assessment including the “SCOTROC” trial (Scottish randomised trial on ovarian cancer comparing carboplatin plus paclitaxel versus carboplatin plus docetaxel), prior to possible licensing for treatment in ovarian cancer. These changes observed in primary treatment have resulted in a skewed grouping of patients on the various chemotherapeutic regimens in this study. However, the results of this investigation indicate that in the subset of patients treated with docetaxel either as a single agent or in combination with a platinum agent, the presence of CYP1B1 in the tumour resulted in a poorer overall survival, supporting the in vitro data. In summary this study provides evidence to support the concept that the presence of CYP1B1 in tumour cells may have an important role in drug resistance, especially to docetaxel.

Cytotoxic Effects of Treatment with Anti-Cancer Drugs

A protein band of approximately 52 kDa was identified in the cellular homogenate from the V79MZh1B1 cell line, corresponding to the expected molecular size observed with lymphoblastoid cells which also express human CYP1B1 [3]. No immunoreactive band was observed in the parental V79MZ cell line. This confirms the presence of CYP1B1 immunoreactivity in the CYP1B1 transfected cells and an absence of CYP1B1 in the parental non-transfected cells.

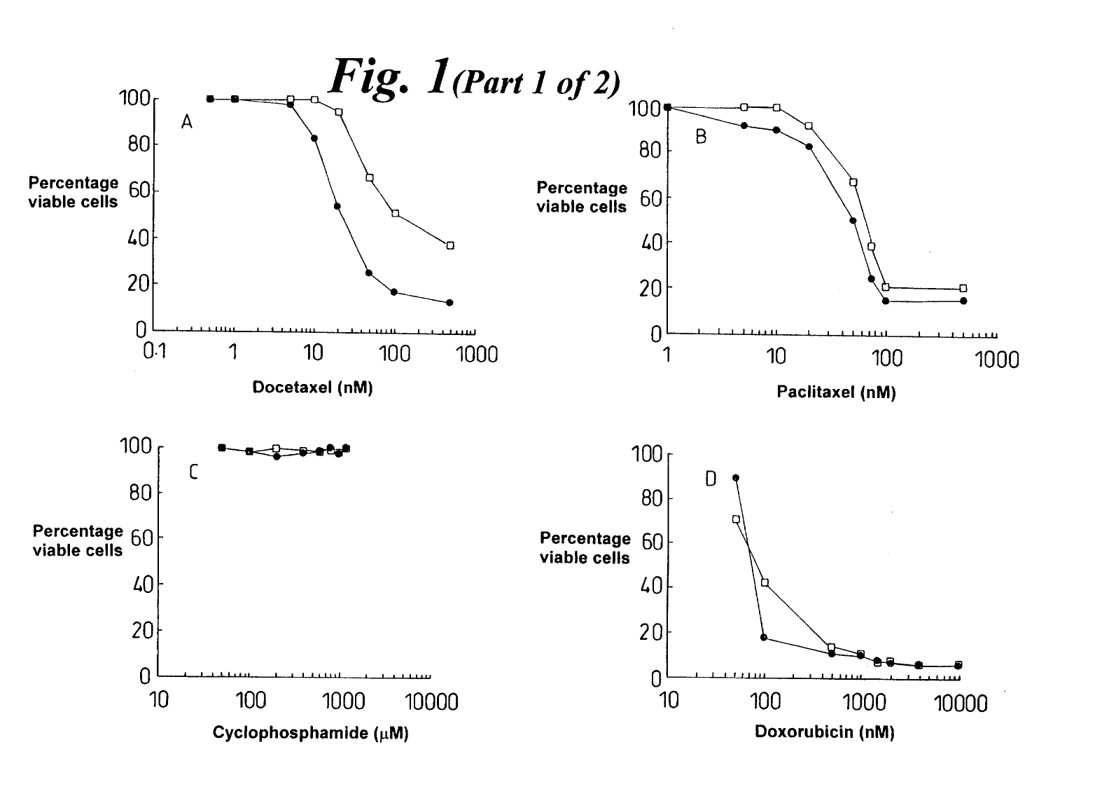

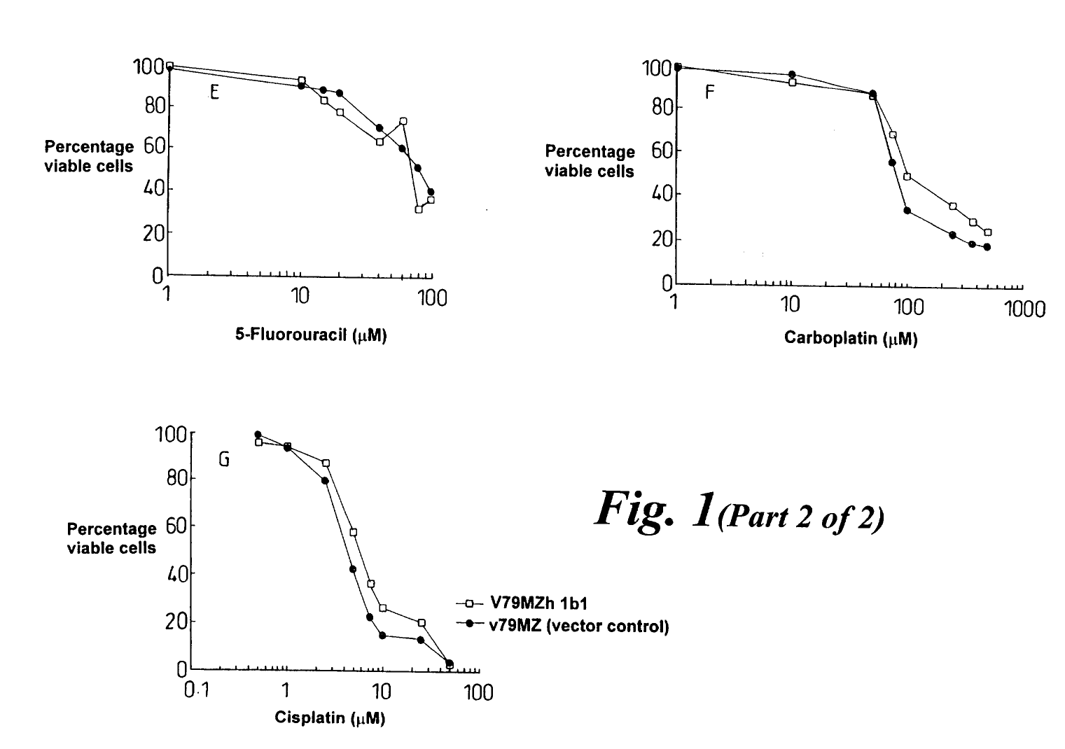

The influence of CYP1B1 on cytotoxicity was evaluated for seven of the fourteen anti-cancer agents investigated. The range of concentrations for each drug used in this study was based on previous experiments with other P450s [23]. A significantly greater (at least four-fold) decreased sensitivity to docetaxel was observed in cells expressing CYP1B1 compared with non-CYP1B1 expressing cells (FIG. 1A. and Table 1). The cytochrome P450 CYP1 inhibitor ANF was used at serial concentrations (1, 10 and 100 μM) to determine if the differential cytotoxicity demonstrated in the cells expressing CYP1B1 was due to metabolism by CYP1B1 (FIG. 2). Neither cell line (parental or CYP1B1 expressing) showed cytotoxicity on exposure to any of the concentrations of ANF. In contrast to the decreased sensitivity observed with docetaxel, no significant difference in cytotoxicity was observed between CYP1B1 expressing and non-expressing cells after exposure to paclitaxel (FIG. 1B. and Table 1). No cytotoxicity was observed in either cell line after exposure to cyclophosphamide (FIG. 1C and Table 1). In addition, no significant difference in cytotoxicity was observed between V79MZ and V79MZh1B1 cells after exposure to doxorubicin, 5-FU, carboplatin or cisplatin (FIGS. 1D-1G; Table 1).

P4501B1 has a Role in Metabolism of Anti-Cancer Agents

The metabolism of anti-cancer drugs by CYP1B1 was studied using an inhibition of EROD assay. Control incubations (with similar amounts of drug vehicle) have shown that inhibition of EROD activity was due to the anti-cancer and test compounds mentioned in the attached results. Ki values were determined by the Dixon's replots: see Table 2. The low Ki values show that CYP1B1 has a strong binding affinity for many anti-cancer agents and could biotransform these drugs.

Many anti-cancer agents showed strong binding affinity (Ki values) on cytochrome 1B1. Moreover, the two anti-cancer agents tested in biotransformation studies, paclitaxel and flutamide, are biotransformed by CYP1B1. Therefore, CYP1B1, which is over-expressed in tumour cells, could have important consequences in the development of new drugs of new therapies and in the prediction of therapy outcome.

Inhibition of CYP1B1

As initial screening for the inhibition of CYP1B1 activity, resorufin production was measured with 1 μM ethoxyresorufin in the presence of 0 μM (as control) or 100 μM anticancer agent. In these conditions, vinblastine, vincristine, 5-fluorouracil, etoposide, and cyclophosphamide did not inhibit CYP1B1 activity. In contrast, flutamide, paclitaxel, mitoxantrone, docetaxel, doxorubicin, daunomycin, and tamoxifen inhibited CYP1B1 activity by decreasing the production of resorufin by 53 to 99%. Further inhibition studies performed with three concentrations of ethoxyresorufin and six concentrations of drugs identified flutamide, mitoxantrone, docetaxel, and paclitaxel as competitive inhibitors with Kᵢ values of 1.0, 11.6, 28.0, and 7.85 μM respectively (Table 2). Noncompetitive or mixed inhibition was observed for daunomycin, doxorubicin, and tamoxifen, and Kᵢ values were 2.1, 2.6, and 5.0 μM, respectively (Table 2).

Similarly, known CYP inhibitors and putative CYP1B1 substrates were also initially screened at 100 μM and agents with an apparent interaction were further characterised as described above. Erythromycin and cyclosporine did not inhibit CYP1B1 activity (10%; Table 2). In contrast, testosterone and estradiol were competitive inhibitors of EROD (Kᵢ=1.9 and 411.8 μM respectively). Potent non-competitive inhibition by ketoconazole (Kᵢ=0.3 μM) and α-naphthoflavone (Kᵢ=2.8 μM) was observed.

Flutamide Metabolism

Flutamide was a potent competitive inhibitor of CYP1B1, suggesting that it is a putative substrate. In vitro incubations of flutamide were performed with human liver microsomes or various cDNA-expressed human CYPs. Two flutamide metabolites were produced in the presence of a NADP-regenerating system. One metabolite was observed after incubation with CYP1B1, CYP1A1, or CYP1A2. This metabolite has been identified as 2-hydroxyflutamide, following metabolic studies of CYP1B1 and CYP1A1 activities. As previously reported (Shet et al, 1997), the production of the other metabolite is via CYP3A4 activity.

The 2-hydroxylation of flutamide was produced by microsomes containing human CYP1B1, CYP1A1, and CYP1A2. Production of 2-hydroxyflutamide by CYP1B1 was described by Michaelis-Menten kinetics, and Kₘ and Vₘₐₓ values were calculated by Eadie-Hofstee plots. Flutamide was a competitive inhibitor of CYP1B1, CYP1A1, and CYP1A2 activities, with Kᵢ values ranging from 1.0 to 10.3 μM. For CYP1B1 and CYP1A2, similar Kₘ and Kᵢ values were obtained. In contrast, Kₘ and Kᵢ values for CYP1A1 are 5 and 10 times higher than CYP1B1, respectively. Vₘₐₓ values for 2-hydroxylation of flutamide are different for all three enzymes, likely reflecting differences in cytochrome c reductase activity in the microsome preparation used. In this study, the cytochrome c reductase activity was 310, 1600, and 2330 mnol/min×mg of proteins for CYP1B1, CYP1A1, and CYP1A2, respectively.

Kidney Tumour Cell Assay

These results of this assay confirms that an inhibitor of CYP1B1 can inhibit the protein when it is present in kidney tumour samples, providing confirmation that the inhibitors can be employed with anti-cancer agents. Three separate tumour samples were used and the CYP1B1 activity in metabolising EROD was measured in the presence and absence of the inhibitor α-naphthoflavone.

Sample 1: CYP1B1 activity 252 fmol/min/mg protein

plus 10 nM ANF 145 fmol/min/mg protein

Sample 2: CYP1B1 activity 993 fmol/min/mg protein

plus 10 nM ANF 470 fmol/min/mg protein

Sample 3: CYP1B1 activity 880 fmol/min/mg protein

plus 10 nM ANF 570 fmol/min/mg protein

Benzpyrene Assay

This assay can be used to measure CYCP1B1 activity in tumour samples, and in particular in frozen sections of tumour. In the assay, fresh, frozen sections of tumour were incubated with benzpyrene (10-100 μM and NADPH for 1 hour at 37° C. and then mounted in alkaline glycerol jelly. The presence of 1B1 was visualised by fluorescence microscopy using excitation at 400-450 nm and detecting emission at 520 nm through a dichroic mirror having a 510 nm frequency cut off. Yellow-green fluorescence is observed in tumour cells.

Discussion

In this study a Chinese hamster cell line which stably expresses human CYP1B1 [21] was used as a bioassay to assess the effect of CYP1B1 on the cytotoxicity of a range of anti-cancer drugs. Although several of the drugs used in this study are clinically relevant in the treatment of ovarian cancer, i.e. cisplatin, carboplatin and 5-FU [30,31], they have no known interactions with cytochrome P450 enzymes. However, these drugs were used to provide controls to assess the validity of the cytotoxicity assay. The pro-drug cyclophosphamide provided an appropriate negative control (i.e. non-cytotoxic to either parental or CYP1B1 expressing cells) as it requires activation by other cytochrome P450 enzymes (CYP2B6 and CYP3A4) before becoming cytotoxic, but we have shown previously that it does not interact with CYP1B1 [32]. Doxorubicin is known to be metabolised to more cytotoxic compounds by the action of other cytochrome P450 enzymes (CYP3A) [20], whereas the taxanes paclitaxel and docetaxel are both metabolised to pharmacologically less active metabolites by cytochrome P450 enzymes; (CYP2C8 and CYP3A4 for paclitaxel and CYP3A4 for docetaxel) [12,13,14,15,33]. Previous studies have demonstrated that V79MZ cells when stably transfected with other cytochrome P450 enzymes have exhibited appropriate cytotoxicity when exposed to individual anti-cancer drugs establishing this cell line as an appropriate model for investigating anti-cancer drug cytotoxicity [23].

It is widely known that both primary cultures and human tumour cell lines rapidly lose the ability to constitutively express cytochrome P450 enzymes in culture. Indeed, we have shown that the MCF-7 human breast carcinoma and PEO4 ovarian adenocarcinoma derived cell lines do not express CYP1B1 (unpublished observations) even though we have shown over-expression of CYP1B1 in both breast and ovarian tumours [1]. The lack of constitutive expression of cytochrome P450s in tumour cell lines was overcome by the use of a stably transfected cell line expressing CYP1B1. CYP1B1 activity in these cells was previously shown to be approximately 10 pmol/min/mg of protein by the EROD assay [21], and is likely to be comparable with that observed in human tumours.

These results show that docetaxel is metabolised by expressed human CYP1B1 [32]. Docetaxel is a semi-synthetic taxane derived from the European yew, and is currently under investigation, for use as first line treatment of ovarian cancer [12-13,34]. The key finding of the current study was the significant differential cytotoxicity observed on exposure to docetaxel, the cytotoxicity observed in CYP1B1 expressing cells was four-fold less than that observed in non-CYP1B1 expressing cells. In addition co-treatment of these cells with the known CYP1 P450 inhibitor ANF [27] resulted in the complete reversal of differential cytotoxicity observed in these cells, i.e. the effect is attributed to metabolism of docetaxel by CYP1B1. The resistance to the cytotoxic effects of docetaxel in those cells expressing CYP1B1 may have important clinical implications.

Inhibition of CYP1B1 in tumours may offer a specific mechanism for overcoming the resistance to docetaxel- and other drugs. Development of a specific inhibitor to CYP1B1 is clinically important as ANF also inhibits CYP1A1 and CYP1A2 [27]. Since CYP1B1 is over-expressed in tumour but not in normal tissue, increasing the tumour sensitivity to anti-cancer drugs by CYP1B1 inhibition would not be expected to have an effect on normal tissues.

In summary, this study provides evidence for the concept that the presence of CYP1B1 in tumour cells may have an important role in drug resistance.

TABLE 1
^{a}IC50 values for V79MZ and V79MZh1B1 cell lines treated with
several anti-cancer drugs

| Drug | V79MZ (control vector) | | V79MZhB1 | | p-value |
|---|---|---|---|---|---|
| Docetaxel | 22 | nM | 100 | nM | 0.03 |
| Paclitaxel | 35 | nM | 60 | nM | NS |
| Cyclophosphamide | NC | | NC | | ND |
| Doxorubicin | 80 | nM | 90 | nM | 0.79 |
| 5-Fluorouracil | 70 | μM | 80 | μM | NS |
| Carboplatin | 80 | μM | 100 | μM | NS |
| Cisplatin | 4.4 | μM | 6 | μM | NS |

Statistical comparison of dose response curves (IC50s) using Mann-Whitney U test.
^{a}IC50 = Drug concentration at which 50% of the cells are viable. The data represent the means of 24 determinations per drug concentration.
NC = No cytotoxicity (no cytotoxicity observed with cyclophosphamide, ND = Not determined, NS = not statistically significant for paclitaxel, 5FU, cisplatin or carboplatin. The statistical software package we used did not provide p values in many cases when the result was nonsignificant.

TABLE 2
Cytochrome 1B1: Ki values
Ki values calculated from Dixon re-plots of EROD activity mediated
by cDNA expressed cytochrome 1B1

| | |
|---|---|
| Inhibition Type | Ki Values |

| | | | |
|---|---|---|---|
| Anticancer Agents | | | |
| Flutamide | competitive | mean | 0.99 μM |
| | | sd | 0.07 μM |
| Tamoxifen | non-competitive | mean | 5.02 μM |
| | | sd | 0.08 μM |
| Mitoxantrone | competitive | mean | 11.63 μM |
| | | sd | 0.03 μM |
| Paclitaxel | competitive | mean | 7.85 μM |
| | | sd | 0.91 μM |
| Docetaxel | competitive | mean | 28.03 μM |
| | | sd | 9.78 μM |
| Doxorubicin | mixed | mean | 2.58 μM |
| | | sd | 0.18 μM |
| Daunomycin | mixed | mean | 2.12 μM |
| | | sd | 0.11 μM |
| Test Compounds | | | |
| α-naphthoflavone | non-competitive | mean | 2.80 nM |
| | | sd | 0.53 nM |
| Testosterone | competitive | mean | 411.83 μM |
| | | sd | 40.38 μM |
| Estradiol | competitive | mean | 1.85 μM |
| | | sd | 0.06 μM |
| Ketoconazole | non-competitive | mean | 0.27 μM |
| | | sd | 0.01 μM |

TABLE 3
Clinicopathological characteristics of patients with ovarian carcinoma

| | |
|---|---|
| Age of Patients | |
| Median (Range) | 63 years (30-89 years) |

| | | |
|---|---|---|
| Stage | | |
| 1 | 44/172 | (25%) |
| 2 | 15/172 | (9%) |
| 3 | 103/172 | (60%) |
| 4 | 10/172 | (6%) |
| Histology | | |
| Serous cystadenocarcinoma | 102/172 | (59%) |
| Endometrioid carcinoma | 35/172 | (21%) |
| Mucinous cystadenocarcinoma | 24/172 | (14%) |
| Clear cell adenocarcinoma | 7/172 | (4%) |
| Malignant mixed Mullerian tumour | 4/172 | (2%) |

TABLE 4
The number of cases showing CYP1B1 expression in the different
histological types of primary ovarian carcinoma

| | CYP1B1 immunoreactivity | | | | |
|---|---|---|---|---|---|
| Diagnosis | Negative | Weak | Moderate | Strong | Total |
| Serous | 8 | 15 | 22 | 54 | 99 |
| cystadenocarcinoma | | | | | |
| Endometrioid | 2 | 7 | 13 | 13 | 35 |
| carcinoma | | | | | |
| Mucinous | 3 | 4 | 4 | 12 | 23 |
| cystadenocarcinoma | | | | | |
| Clear cell | 1 | 3 | 0 | 3 | 7 |
| adenocarcinoma | | | | | |
| Malignant mixed | 0 | 0 | 0 | 3 | 3 |
| Mullerian tumour | | | | | |
| Total | 14 | 29 | 39 | 85 | 167 |

TABLE 5
The number of cases of CYP1B1 expression in the different
histological types of metastatic epithelial ovarian carcinoma

| | CYP1B1 immunoreactivity | | | | |
|---|---|---|---|---|---|
| Diagnosis | Negative | Weak | Moderate | Strong | Total |
| Serous | 2 | 4 | 20 | 13 | 39 |
| cystadenocarcinoma | | | | | |
| Endometrioid | 0 | 1 | 0 | 4 | 5 |
| carcinoma | | | | | |
| Mucinous | 0 | 0 | 2 | 0 | 2 |
| cystadenocarcinoma | | | | | |
| Malignant mixed | 1 | 0 | 0 | 1 | 2 |
| Mullerian tumour | | | | | |
| Total | 3 | 5 | 22 | 18 | 48 |

### REFERENCES

The references cited herein are all expressly incorporated by reference.

[1] Murray et al. Tumour-specific expression of cytochrome P450 CYP1B1. Cancer Res 1997;57:3026-31.

[2] Murray et al. The regulation, function and tissue specific expression of cytochrome P450 CYP1B1. Annu Rev Pharmacol Toxicol 2001 (in press).

[3] McFadyen et al. Immunohistochemical localisation of cytochrome P450 CYP1B1 in breast cancer with monoclonal antibodies specific for CYP1B1. J Histochem Cytochem 1999;47:1457-64.

[4] McKay et al. Expression of cytochrome P450 CYP1B1 in breast cancer. FEBS Letters 1995;374:270-72.

[5] Hayes et al. 17b-Estradiol hydroxylation catalysed by human cytochrome P450 1B1. Proc Natl Acad Sci USA 1996;93:9776-81.

[6] Liehr et al. 4-Hydroxylation of estradiol by human uterine myometrium and myoma microsomes: implications for the mechanism of uterine tumourigenesis. Proc Natl Acad Sci USA 1995;92:9220-24.

[7] Spink et al. The effects of 2,3,7,8-tetrachlorodibenzo-p-dioxin on estrogen metabolism in MCF-7 breast cancer cells: evidence for induction of a novel 17b-estradiol 4-hydroxylase. J Steroid Biochem Molec Biol. 1994;51:251-58.

[8] Liehr et al. 4-Hydroxylation of estrogens as a marker of human mammary tumours. Proc Natl Acad Sci USA 1996;93:3294-96.

[9] Le-Blanc et al. Interaction of anticancer drugs with hepatic monooxygenase enzymes. Drug Metab Rev 1989;20:395-439.

[10] Kivisto et al. The role of human cytochrome P450 enzymes in the metabolism of anti-cancer agents: implications for drug interactions. Br J Clin Pharmacol 1995;40:523-30.

[11] Iyer et al. Pharmacogenetics and cancer chemotherapy. Eur J Cancer 1998;34:1493-99.

[12] Marre et al. Hepatic biotransformation of docetaxel (Taxotere) in vitro: involvement of the CYP3A subfamily in humans. Cancer Res 1996;56:1296-302.

[13] Crommentuyn et al. In-vitro metabolism of anti-cancer drugs, methods and applications: paclitaxel, docetaxel, tamoxifen and ifosfamide. Cancer Treat Rev 1998;24:345-66.

[14] Sonnichsen et al. Clinical pharmacokinetics of paclitaxel. Clin Pharmacokinet 1994;27:256-69.

[15] Rahman et al. Selective biotransformation of taxol to 6a-hydroxytaxol by human cytochrome P450 CYP2C8. Cancer Res 1994;54:5543-46.

[16] Harris et al. Isolation structural determination, and biological activity of 6a-hydroxytaxol, the principle human metabolite of taxol. J Med Chem 1994;37:706-9.

[17] Chang et al. Differential activation of cyclophosphamide and ifosphamide by cytochromes P-450 2B and 3A in human liver microsomes. Cancer Res 1993;53:5629-37.

[18] Bohnenstengel et al. Characterization of the cytochrome P450 involved in side-chain oxidation of cyclophosphamide in humans. Eur J Clin Pharmacol 1996;51:297-301.

[19] Goeptar et al. Cytotoxicity of mitomycin C and adriamycin in freshly isolated rat hepatocytes: the role of cytochrome P450. Cancer Res 1994;54:2411-18.

[20] Lewis et al. Role of cytochrome P450 from the human CYP3A gene family in the potentiation of morphilino doxorubicin by human liver microsomes. Cancer Res 1992;52:4379-84.

[21] Luch et al. Stable expression of human cytochrome P450 1B1 in V79 Chinese hamster cells and metabolically catalyzed DNA adduct formation of dibenzo[a,l]pyrene. Chem Res Toxicol 1998;11: 686-95.

[22] Engst et al. Benzylic hydroxylation of 1-methylpyrene and 1-ethylpyrene by human and rat cytochromes P450 individually expressed in V79 Chinese hamster cells. Carcinogenesis 1999;20:1777-85.

[23] Philip et al. Use of V79 cells with stably transfected cytochrome P450 cDNAs in studying the metabolism and effects of cytotoxic drugs. Cancer Chemother Pharmacol 1999;43:59-67.

[24] Doehmer. V79 Chinese hamster cells genetically engineered-for cytochrome P450 and their use in mutagenicity and metabolism studies. Toxicology 1993;82:105-118.

[25] Bradford. A rapid and sensitive method for the quantitation of microgram quantities of protein utilizing the principle of protein-dye binding. Anal Biochem 1976;72:248-54.

[26] Crespi et al. Development of a human lymphoblastoid cell line constitutively expressing human CYP1B1 cDNA: substrate specificity with model substrates and promutagens. Mutagenesis 1997;12:83-89.

[27] Shimada et al. Selectivity of polycyclic inhibitors for human cytochrome P450s 1A1, 1A2, and 1B1. Chem Res Toxicol 1998;11:1048-56.

[28] Plumb et al. Effects of the pH dependence of 3-(4,5-dimethylthiazol-2-yl)-2,5-diphenyl-tetrazolium bromide-formazan absorption on chemosensitivity determined by a novel tetrazolium based assay. Cancer Res 1989;49:4435-40.

[29] Kuilman et al. Cytochrome P450 mediated metabolism and cytotoxicity of aflotoxin B₁ in bovine hepatocytes. Toxicol in vitro 2000;14:321-27.

[30] Adams et al. Chemotherapy for ovarian cancer-a consensus statement on standard practice. Br J Cancer 1998;78:1404-06.

[31] Morgan et al. Phase II trial of combination intraperitoneal cisplatin and 5-fluorouracil in previously treated patients with advanced ovarian cancer: long-term follow-up. Gynecol Oncol 2000;77:433-38.

[32] Rochat et al. Human CYP1B1 and anti-cancer agent metabolism: mechanism for tumour specific drug interaction? J Pharmacol Exp Ther 2001;296: 537-541.

[33] Shou et al. Role of human cytochrome P450 3A4 and 3A5 in the metabolism of taxotere and its derivatives: enzyme specificity, interindividual distribution and metabolic contribution in human liver. Pharmacogenetics 1998; 8:391-401.

[34] Crown et al. The taxanes: an update. The Lancet 2000;355:1176-78.

1. Use of a substance capable of inhibiting CYP1B1 protein and an anti-cancer drug for the preparation of a medicament for the treatment of cancer.

2. The use of claim 1, wherein the substance capable of inhibiting CYP1B1 protein inhibits an activity of CYP1B1 enzyme in metabolising or inactivating the anti-cancer drug or a pro-drug form thereof, or a metabolic product of the anti-cancer drug or pro-drug.

3. The use of claim 2, wherein CYP1B1 causes a loss of cytotoxicity of the anti-cancer drug, or in the case of a pro-drug, the capacity to be converted to active drug.

4. The use of any one of the preceding claims, wherein the substance capable of inhibiting CYP1B1 protein is a flavonoid, a flavone or ethenyl pyrene.

5. The use of claim 4, wherein the substance is α-naphthoflavone.

6. The use of any one of the preceding claims, wherein the anti-cancer drug is a docetaxel, paclitaxel, flutamide, tamoxifen, mitoxantrone, doxorubicin or daunomycin.

7. The use of any one of the preceding claims, wherein the medicament is used for the treatment of breast cancer, kidney cancer, colorectal cancer, prostate cancer, liver cancer or ovarian cancer.

8. The use of any one of the preceding claims, wherein the substance capable of inhibiting CYP1B1 protein and the anti-cancer agent are formulated together in a composition.

9. The use of any one of claims 1 to 7, wherein the substance capable of inhibiting CYP1B1 protein and the anti-cancer agent are formulated for sequential administration.

10. A composition comprising a substance capable of inhibiting CYP1B1 protein and an anti-cancer drug, in combination with a physiologically acceptable carrier.

11. The composition of claim 10, wherein the substance capable of inhibiting CYP1B1 protein inhibits an activity of CYP1B1 enzyme in metabolising or inactivating the anti-cancer drug or a pro-drug form thereof, or a metabolic product of the anti-cancer drug or pro-drug.

12. The composition of claim 11, wherein CYP1B1 causes a loss of cytotoxicity of the anti-cancer drug, or in the case of a pro-drug, the capacity to be converted to active drug.

13. The composition of any one of claims 10 to 12, wherein the substance capable of inhibiting is a flavonoid, a flavone or ethenyl pyrene.

14. The composition of claim 13, wherein the substance is α-naphthoflavone.

15. The composition of any one of claims 10 to 14, wherein the anti-cancer drug is docetaxel, paclitaxel, flutamide, tamoxifen, mitoxantrone, doxorubicin or daunomycin.

16. A kit comprising:
(a) in a first container, a substance capable or inhibiting CYP1B1 protein; and,
(b) in a second container, an anti-cancer drug,
wherein the substances are formulated with a physiologically acceptable carrier and are for simultaneous or sequential administration.

17. A method for treating cancer, the method comprising administering to a patient in need a combination of a substance capable of inhibiting CYP1B1 protein and an anti-cancer drug.

18. A method of screening for CYP1B1 inhibitors for use in combination with anti-cancer drugs, the method comprising:
(a) contacting a candidate substance with CYP1B1 protein under conditions where the candidate substance and CYP1B1 can interact;
(b) measuring the activity of the CYP1B1 protein and comparing the value obtained to standards;
(c) selecting a candidate substance which has the effect of inhibiting CYP1B1; and
(d) testing the candidate substance in combination with CYP1B1 and one or more anti-cancer drugs to determine whether it is capable of reducing the effect of CYP1B1 in inactivating or metabolising the anti-cancer drug.

19. The method of claim 18, wherein the testing step employs an in vitro interaction assays or a cell based assays measuring the effect of the candidate substance in reducing the loss of cytotoxicity of the anti-cancer drugs caused by CYP1B1 action.

20. A method of determining the diagnosis, prognosis or responsiveness to treatment of a patient having ovarian cancer, the method comprising determining a presence or amount of CYP1B1 protein in a sample from a patient comprising ovarian cancer cells and correlating the presence or amount to control values.
